(19) United States
(12) Patent Application Publication
(10) Pub. No.: US 2006/0135383 A1
(43) Pub. Date: Jun. 22, 2006

(54) Cleansing compositions comprising polymeric emulsifiers and methods of using same

(76) Inventors: Anthony J. Cossa, Branchburg, NJ (US); Russel M. Walters, Philadelphia, PA (US); Joseph LiBrizzi, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/016,036

(22) Filed: Dec. 17, 2004

(51) Int. Cl. A61K 8/00

(52) U.S. Cl. 510/130

(57) ABSTRACT

Provided are compositions comprising at least one polymeric emulsifier, at least one hydrophobic agent, and at least one foaming agent, the compositions exhibiting at least one of the properties selected from the group consisting of a Deposition Value of greater than about 7 micrograms per square centimeter, a Maximum Foam Volume of greater than about 100 mL, and combinations of two or more thereof. Also provided are methods of cleaning the skin using the compositions, and methods of promoting products comprising the compositions having high foaming and/or high deposition properties.

### FIELD OF THE INVENTION

The present invention relates to compositions comprising polymeric emulsifiers and, more particularly, to emulsion compositions comprising polymeric emulsifiers which exhibit unexpected deposition and foaming properties.

### BACKGROUND

Conventional surfactant based cleansers effectively remove oils and dirt from skin, however, applicants have noted that they tend to leave skin dry or irritated because the surfactants tend to emulsify and remove oils that are naturally present on the surface of skin. Accordingly, when utilizing these cleansing systems, it is often necessary to apply a conditioning product, such as a lotion, to the skin in an attempt to replace the natural skin oil or emollients and moisture removed by the process of cleansing.

A conventional approach to solving the above-mentioned problem has been to develop a single “2-in-1” type cleansing system that both cleans and conditions in one step. Many such compositions have been described in the prior art such as for example U.S. Pat. No. 6,113,892 (Newell et al.), U.S. patent application 2003/018503 (Maubru et al.) and U.S. Pat. No. 5,085,857 (Reid et al.). These compositions typically possess high foam and rely on the cationic nature of the conditioner to provide substantivity to the skin. Unfortunately, applicants have noted that these compositions have rather low efficiency of conditioner deposition due to the high loads of surfactant, particularly the high loads of foaming surfactants.

Other conventional approaches to the problem include those taught by U.S. Pat. No. 6,696,068 (Crotty et al.) and U.S. Pat. No. 2003/0068342A1 (Crotty et al.) which describe the use of a silicone copolyol sulfosuccinate in combination with a silicate and crosslinked carboxyvinyl polymer. Another example can be found in U.S. Pat No. 5,336,497 (Guerrero et al.) which discloses the use of a silicone copolyol sulfosuccinate in combination with a betaine. Yet another example is disclosed in U.S. Pat. No. 5,236,710 (Guerrero et al.) which describes a silicone copolyol sulfosuccinate in combination with an emulsifying copolymer to achieve conditioning of skin. In each case, applicants note that such compositions suffer from little to no deposition of hydrophobic agents on to the skin.

Yet another approach to the problem of replacing emollients on skin lost to cleansing is to formulate emulsion type cream cleansers having high loads of emollients incorporated together with surfactants to emulsify the emollient (in order to provide shelf stability) and provide some foam in use. For example, U.S. Pat. No. 2002/0076422A1 (Shah et al.) describes a water-based cream cleanser system containing a high level of emollients and glycerin in combination with surfactants. U.S. Pat. No. 5,409,706 (Ramirez et al.) teaches an anhydrous cream cleanser system containing a high level of emollients and glycerin in combination with surfactants. Although these technologies attempt to solve the problem of re-depositing emollients lost during cleansing, they are inefficient due to the stripping and emulsification of emollients caused by the high surfactant load.

In an apparent attempt to enhance deposition of emollients, emulsion cleanser systems that are devoid or contain minimal surfactant have been recognized in the art. Recently, polymers have been developed which can stabilize oil droplets in a water-based system without the use of surfactant emulsifiers. U.S. Pat. No. 5,373,044 (Adams et al.) describes a polymer of at least one olefinically unsaturated carboxylic acid containing at least one activated carbon-to-carbon olefinic double bond and at least one carboxyl group which has both thickening and emulsifying/dispersing properties. U.S. Pat. No. 6,489,395 (Loffler and U.S. Pat. No. 6,682,750 (Loffler et al.) describes crosslinked water-soluble or water-swellable copolymers based on acrylamidoalkylsulfonic acids and cyclic N-vinylcarboxamides and/or linear N-vinylcarboxamides. U.S. Pat. No. 5,004,598 (Lockhead et al.) describes a method for producing lotions utilizing a copolymer of acrylic acid and long chain acrylate as the polymeric.

Further formulation refinements of polymeric emulsifiers are described, such as in U.S. Pat. No. 6,620,420 (Lanzendorfer et al.) and U.S. patent application No. 2004/0037797A1 (Nielsen et al.) which describe the use of ammonium acryloyldimethyltaurate/vinylpyrrolidone copolymers in combination with lipids and emulsifiers to generate emulsions that are oil-in water and water-in-oil, respectively. These documents, however, do not provide rinsable compositions that provide both foam and deposition.

Yet another approach is described in U.S. patent application (Deckner et. al.) in which rinseable skin conditioning compositions comprise high internal phase emulsions and stabilizers. These compositions are, however, substantially free of surfactant and are not capable of providing adequate foam.

To overcome such disadvantages, applicants have recognized that it would be advantageous to develop a composition that possessed adequate foam levels and could deposit high loads of emollient or other hydrophobic agent on the skin, wherein the emollient or hydrophobic agent remains behind on skin after cleansing with the composition.

### SUMMARY OF THE INVENTION

According to one aspect of the invention, provided are compositions comprising at least one polymeric emulsifier, at least one hydrophobic agent, and at least one foaming agent, the compositions exhibiting at least one of the properties selected from the group consisting of a Deposition Value of greater than about 7 micrograms per square centimeter, a Maximum Foam Volume of greater than about 100 mL, and combinations of two or more thereof.

In another aspect, provided is a method of cleansing skin comprising applying a composition of the present invention to the skin.

In yet another aspect, provided is a method of promoting a product comprising providing a product comprising a composition of the present invention and directing a user to apply the product to the skin or hair to achieve the high foaming and/or high deposition properties associated with the product.

### DESCRIPTION OF PREFERRED EMBODIMENTS

Applicants have discovered unexpectedly that polymeric emulsifiers, hydrophobic agents, and foaming agents can be combined to form compositions that overcome one or more of the disadvantages associated with conventional cleansers. That is, applicants have recognized that the emulsion compositions of the present invention tend to exhibit unexpectedly high deposition of hydrophobic agents on the skin as well as foaming as compared to conventional cleansing compositions.

In particular, applicants have tested the deposition properties associated with compositions of the present invention via the “Deposition Test”, described in detail below, which measures the “Deposition Value” (related to the deposition of hydrophobic agents on the skin wherein a higher Deposition Value represents the desirable property of high deposition of hydrophobic agent) of compositions of the present invention. Additionally, applicants have tested the foaming properties associated with compositions of the present invention via the “Foam Test”, described in detail below, which measures the “Maximum Foam Volume” (related to the ability of the composition to provide foam wherein a higher Maximum Foam Volume represents the desirable property of high foaming) of the compositions of the present invention. Applicants have discovered unexpectedly that the compositions of the present invention exhibit Deposition Values as well as Maximum Foam Values that are unexpectedly high as compared to conventional compositions.

For example, in certain embodiments, the present compositions exhibit a Deposition Value that is preferably greater than about 7 micrograms per square centimeter, more preferably greater than about 15 micrograms per square centimeter, even more preferably greater than about 26 micrograms per square centimeter, even more preferably greater than about 30 micrograms per square centimeter, and most preferably greater than about 34 micrograms per square centimeter. Furthermore, applicants have unexpectedly discovered that it is also possible to achieve Maximum Foam Volumes that are preferably greater than about 100 milliliters (mL), more preferably greater than about 400 mL, and even more preferably greater than about 600 mL, such as between about 600 mL and about 800 mL.

Furthermore, applicants have unexpectedly discovered that is possible to achieve both high foam levels while simultaneously providing high levels of deposition. For example, in certain embodiments, the present compositions exhibit a foam level that is greater than about 400 mL and a deposition greater than about 7 micrograms per square centimeter, more preferably a foam level that is greater than about 400 mL and a deposition greater than about 15 micrograms per square centimeter, even more preferably a foam level that is greater than about 400 mL and a deposition greater than about 26 micrograms per square centimeter, even more preferably a foam level that is greater than about 600 mL and a deposition greater than about 26 micrograms per square centimeter, even more preferably a foam level that is greater than about 600 mL and a deposition greater than about 30 micrograms per square centimeter, and most preferably a foam level that is greater than about 600 mL and a deposition greater than about 33 micrograms per square centimeter. Applicants have recognized that such significant properties and combinations thereof are not only unexpected, but may also be used to significant advantage in a variety of applications.

According to certain preferred embodiments, the compositions of the present invention include a salt-sensitive polymeric emulsifier, a hydrophobic agent, and a foaming agent. These various classes of components are described below in detail in this specification.

As used herein, the term “polymeric emulsifier” refers generally to a polymer having both hydrophilic and hydrophobic moieties that is capable of contributing to the formation of a stable emulsion between an oil phase and an aqueous phase. Any of a variety of suitable polymeric emulsifiers may be used according to the present invention. In certain preferred embodiments, applicants have recognized it is desirable to use one or more polymeric emulsifiers which tends to provide shelf stability to the composition into which it is added and/or tends to facilitate the deposition of hydrophobic agent onto a substrate and/or tends to support the ability of the composition to provide foam, such as when using the composition to cleanse the skin. In addition, certain preferred polymeric emulsifiers comprise those compounds that are water-soluble and are capable of forming a phase stable emulsion, preferably stable for at least about 1 week, more preferably at least about a month, of a hydrophobic agent of the present invention in water. (As used herein a material is defined as “water-soluble”, if it is possible to form a clear solution by adding only 0.5% by weight of the material in deionized water that is stable at room temperature (no settling or phase-instability) for 48 hours.) Polymeric emulsifiers of the present invention are preferably salt-sensitive, in that, their solubility in water is reduced, often dramatically, in the presence of electrolytes (such as electrolytes typically present as impurities in surfactant cleansing systems and on the surface of skin). A polymeric emulsifier is defined as “salt-sensitive” if it loses its ability to remain phase stable in aqueous solution when sodium chloride has been added. Specifically, a “salt sensitive” polymeric emulsifier will show phase separation and/or a 30% or more change in viscosity (measured using a Brookfield viscometer with an LVT2 spindle at 12 RPM) if 3% sodium chloride is added to a homogenous solution of 1% (active) polymeric emulsifier in deionized water.

The polymers for use as polymeric emulsifiers in the present invention may be of any suitable molecular weight. In certain embodiments of the invention, the polymeric emulsifier has a weight average molecular weight that is preferably greater than about 500,000, more preferably greater than about 250,000, and even more preferably greater than about 100,000.

Polymeric emulsifiers suitable for the present invention may comprise an associative polymer, i.e., a polymer formed from monomers such that individual repeat units are hydrophilic, such as may be formed by addition polymerization of such as acids as acrylic, methacrylic, maleic, itaconic, and the like or combinations to form copolymers thereof. Other suitable polymeric emulsifiers include naturally derived polymers such as polysaccharides that may be derivatized with hydrophobic moieties. Suitable hydrophilic groups include hydroxyl, carboxyl, sulphate, sulphonate, taurate, phosphates, phosphonates, amides, amines and the like. In order to provide hydrophobic character, such polymers may at least partially hydrophobically modified, such as by copolymerizing the polymer with hydrocarbons having a carbon chain length of at least about 3.

Notable commercially available polymeric emulsifiers include, but are not limited to, hydrophobically modified polyacrylic acid commercially under the tradename Pemulen® TR-1 and TR-2 by Noveon, Inc., water-soluble or water-swellable copolymers based on acrylamidoalkyl sulfonic acid and cyclic N-vinylcarboxamides commercially available under the tradename Aristoflex® AVC by Clariant Corporation; water-soluble or water-swellable copolymers based on acrylamidoalkyl sulfonic acid and hydrophobically modified methacrylic acid commercially available under the tradename Aristoflex® HMB by Clariant Corporation and a homopolymer of acrylamidoalkyl sulfonic acid commercially available under the tradename Granthix APP by Grant Industries, Inc.

Another class of notable polymeric emulsifier includes hydrophobically-modified, crosslinked, anionic acrylic copolymers, including random polymers, but may also exist in other forms such as block, star, graft, and the like. In one embodiment, the hydrophobically modified, crosslinked, anionic acrylic copolymer may be synthesized from at least one acidic monomer and at least one hydrophobic ethylenically unsaturated monomer. Examples of suitable acidic monomers include those ethylenically unsaturated acid monomers that may be neutralized by a base. Examples of suitable hydrophobic ethylenically unsaturated monomers include those that contain a hydrophobic chain having a carbon chain length of at least about 3 carbon atoms.

Other materials that may be suitable polymeric emulsifiers include ethylene oxide/propylene oxide block copolymers, sold under the trade name PLURONIC, available from BASF corporation of Parsippany, N.J., modified cellulose polymers such as those modified cellulose polymers described by the trade name KLUCEL, available from Hercules Corporation of Wilmington, Del.

Particularly notable embodiments of the invention are compositions that include hydrophobically modified polyacrylic acid, acrylamidoalkyl sulfonic acid, cyclic N-vinylcarboxamides, acrylamidoalkyl sulfonic acid, hydrophobically modified methacrylic acid, a homopolymer of acrylamidoalkyl sulfonic acid, or combinations thereof as polymeric emulsifiers; and monomeric anionic surfactants, monomeric amphoteric surfactants, or combinations thereof as foaming agents. More particularly notable embodiments of the invention are compositions that include hydrophobically modified polyacrylic acid; water-soluble or water-swellable copolymers based on acrylamidoalkyl sulfonic acid, cyclic N-vinylcarboxamides; water-soluble or water-swellable copolymers based on acrylamidoalkyl sulfonic acid, hydrophobically modified methacrylic acid; a homopolymer of acrylamidoalkyl sulfonic acid, or combinations thereof as polymeric emulsifiers, and include a betaine as the foaming surfactant. Especially notable embodiments of the invention are compositions that include copolymers based on acrylamidoalkylsulfonic acids and cyclic N-vinylcarboxamides and/or linear N-vinylcarboxamides (e.g., Aristoflex® AVC and Aristoflex®V HMB from Clariant Corporation) as polymeric emulsifiers and a betaine as foaming surfactant.

Any suitable amounts of polymeric emulsifier may be used in the compositions of the present invention. In certain preferred embodiments, the compositions of the present invention comprise at least about 0.3 weight percent, for example, between about 0.3% and about 3%, between about 0.3% and about 2%, and between about 0.3% and about 1%.

As used herein and throughout the application, all percents represent percent by weight of the active ingredient (i.e., carriers such as water and impurities are not included in these percentages) based on the total weight of composition, unless otherwise indicated. Also, when it is stated that a particular category of ingredient, e.g., polymeric emulsifier is “present in a weight percentage” that is in a certain range, this implies that the sum total concentration of all ingredients that are classified as polymeric emulsifiers are present in that present range.

Any of a variety of hydrophobic materials that are water-insoluble, are suitable for use herein as a hydrophobic agent. (As used herein, the term “water-insoluble” refers to a material that when added to deionized water to a concentration by weight of 0.5% (with no other additives) at room temperature, cannot be made to form a clear homogeneous mixture for a period of time lasting at least 48 hours.)

Examples of suitable hydrophobic agents include, but are not limited to emollients such as mineral oils, petrolatum, vegetable oils (glyceryl esters of fatty acids, triglycerides), waxes and other mixtures of esters, not necessarily esters of glycerol; polyethylene and non-hydrocarbon based oils such as dimethicone, silicone oils, silicone gums, and the like. In certain embodiments, preferred emollient hydrophobic agents include mineral oil, petrolatum, silicone oils, and combinations thereof.

In another embodiment of the invention, the hydrophobic agent may be a benefit agent. What is meant by a “benefit agent” is an element, an ion, a compound (e.g., a synthetic compound or a compound isolated from a natural source) or other chemical moiety in solid (e.g. particulate), liquid, or gaseous state and compound that has a cosmetic or therapeutic effect on the skin.

Examples of suitable hydrophobic benefit agents include those that provide benefits to the skin, such as, but not limited to, depigmentation agents; reflectants; amino acids and their derivatives; antimicrobial agents; allergy inhibitors; anti-acne agents; anti-aging agents; anti-wrinkling agents, antiseptics; analgesics; shine-control agents; antipruritics; local anesthetics; anti-hair loss agents; hair growth promoting agents; hair growth inhibitor agents, antihistamines; antiinfectives; anti-inflammatory agents; anticholinergics; vasoconstrictors; vasodilators; wound healing promoters; peptides, polypeptides and proteins; deodorants and anti-perspirants; medicament agents; skin firming agents, skin lifting and firming agents; vitamins; skin lightening agents; skin darkening agents; antifungals; depilating agents; counterirritants; insecticides; enzymes for exfoliation or other functional benefits; enzyme inhibitors; poison ivy products; poison oak products; burn products; anti-diaper rash agents; prickly heat agents; vitamins; herbal extracts; vitamin A and its derivatives; flavenoids; sensates; anti-oxidants; sunscreens; anti-edema agents, neo-collagen enhancers, chelating agents; antisebhorreic dermatitis/psoriasis agents; keratolytics; and mixtures thereof.

In certain embodiments, preferred hydrophobic benefit agents include anti-acne agents such as salicylic acid and benzoyl peroxide; retinoids such as retinol; skin lifting agents such as alkanolamines including dimethyl amino ethanol (DMAE); skin lightening agents such as soy extracts; anti-inflammatory agent such as tocopherols including vitamin E, extracts of Feverfew; an anti-wrinkle agents such as retinoids; among other benefit agents.

Any suitable amounts of hydrophobic agent or combinations thereof may be used in the compositions of the present invention. In certain embodiments, the hydrophobic agent is present in the composition in a concentration that is at least about 0.1 weight percent, such as from about 0.1% to about 20%, preferably from about 1% to about 20%, and even more preferably from about 2% to about 15%.

In certain preferred embodiments, the polymeric emulsifier and the hydrophobic agent are present in a respective weight ratio from about 60:1 to about 1:150, preferably from about 40:1 to about 1:120, and more preferably from about 20:1 to about 1:100.

Any material suitable for providing foam in use and also allowing the hydrophobic agent of the composition to tend to remain on the substrate (for example by avoiding re-emulsification of the hydrophobic agent) may be used as a foaming agent of the present invention.

Suitable foaming agents preferably comprise monomeric surfactants. The foaming agent may be a cationic, anionic, non-ionic surfactant that is water soluble and may have a hydrophilic-lipophilic balance (HLB), that is, for example greater than about 10.

Examples of suitable non-ionic monomeric surfactant foaming agents include for example, fatty alcohol acid or amide alkoxylates (including polyoxyalkynated alcohols, alcohol alkoxylates including mixed coconut-oil derived, tallow derived, and synthetic straight-chain—primary, random, or secondary; and polyoxyalkynated-alkylphenols (“alkylphenol alkoxylates”), monoglyceride ethoxylates, sorbitan ester ethoxylates and alkyl polyglycoside. A preferred nonionic surfactant is comprised of decyl glucoside. Decyl Glucoside is the product obtained from the condensation of decyl alcohol with a glucose polymer that conforms generally to the following formula, C₁₆H₃₂O₆, and is commercially available from Cognis Corporation, Pennsylvania under the tradename, “Plantaren 2000.”

Examples of suitable cationic foaming surfactants include alkyl quaternaries (mono, di, or tri), benzyl quaternaries, ester quaternaries, ethoxylated quaternaries, alkyl amines, and mixtures thereof, wherein the alkyl group has from about 6 carbon atoms to about 30 carbon atoms, with about 8 to about 22 carbon atoms being preferred.

Examples of suitable anionic surfactants include those selected from the following classes of surfactants:

an alkyl sulfate of the formula
R′—CH₂OSO₃X′;

an alkyl ether sulfate of the formula
R′(OCH₂CH₂)ᵥOSO₃X′;

an alkyl monoglyceryl ether sulfate of the formula

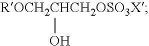

an alkyl monoglyceride sulfate of the formula

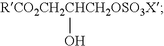

an alkyl monoglyceride sulfonate of the formula

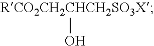

an alkyl sulfonate of the formula
R′—SO₃X′;

an alkylaryl sulfonate of the formula

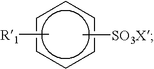

an alkyl sulfosuccinate of the formula:

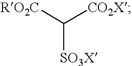

an alkyl ether sulfosuccinate of the formula:

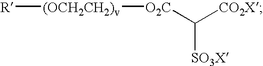

an alkyl sulfosuccinamate of the formula:

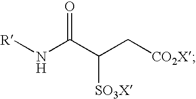

an alkyl amidosulfosuccinate of the formula

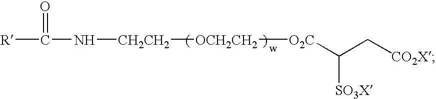

an alkyl carboxylate of the formula:
R′—(OCH₂CH₂)_{w}—OCH₂CO₂X′;

an alkyl amidoethercarboxylate of the formula:

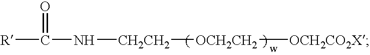

an alkyl succinate of the formula:

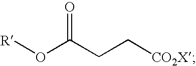

a fatty acyl sarcosinate of the formula:

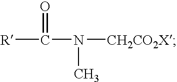

a fatty acyl amino acid of the formula:

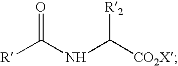

a fatty acyl taurate of the formula:

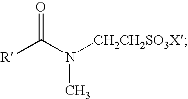

a fatty alkyl sulfoacetate of the formula:

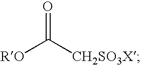

an alkyl phosphate of the formula:

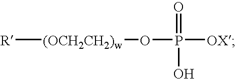

wherein
R′ is an alkyl group having from about 7 to about 22, and preferably from about 7 to about 16 carbon atoms,
R′₁ is an alkyl group having from about 1 to about 18, and preferably from about 8 to about 14 carbon atoms,
R′₂ is a substituent of a natural or synthetic I-amino acid,
X′ is selected from the group consisting of alkali metal ions, alkaline earth metal ions, ammonium ions, and ammonium ions substituted with from about 1 to about 3 substituents, each of the substituents may be the same or different and are selected from the group consisting of alkyl groups having from 1 to 4 carbon atoms and hydroxyalkyl groups having from about 2 to about 4 carbon atoms and
v is an integer from 1 to 6;
w is an integer from 0 to 20;
   and mixtures thereof.

In one embodiment, the anionic surfactant is comprised of disodium laureth sulfosuccinate. Disodium laureth sulfosuccinate is the disodium salt of an ethoxylated lauryl alcohol half ester of sulfosuccinic acid that conforms generally to the following formula, (C₂H₄O)ₙC₁₆H₃₀O₇S.2Na, where n has a value between 1 and 4, and is commercially available from McIntyre Group Ltd. of University Park, Ill. under the tradename, “Mackanate EL.”

In another embodiment, the anionic surfactant is a mixture comprised of sodium methyl 2-sulfolaurate and disodium 2-sulfolaurate and is commercially available from Stepan Company of Northfield, Ill. under the tradename, “Alpha-Step PC48.”

Examples of suitable amphoteric surfactants include, but are not limited to, amphocarboxylates such as alkylamphoacetates (mono or di); alkyl betaines; amidoalkyl betaines; amidoalkyl sultaines; amphophosphates; phosphorylated imidazolines such as phosphobetaines and pyrophosphobetaines; carboxyalkyl alkyl polyamines; alkylimino-dipropionates; alkylamphoglycinates (mono or di); alkylamphoproprionates (mono or di),); N-alkyl β-aminoproprionic acids; alkylpolyamino carboxylates; and mixtures thereof.

Examples of suitable amphocarboxylate compounds include those of the formula:
A-CONH(CH₂)ₓN⁺R₅R₆R₇

wherein

A is an alkyl or alkenyl group having from about 7 to about 21, e.g. from about 10 to about 16 carbon atoms;

x is an integer of from about 2 to about 6;

R₅ is hydrogen or a carboxyalkyl group containing from about 2 to about 3 carbon atoms;

R₆ is a hydroxyalkyl group containing from about 2 to about 3 carbon atoms or is a group of the formula:
R₈—O—(CH₂)ₙCO₂⁻
wherein
R₈ is an alkylene group having from about 2 to about 3 carbon atoms and n is 1 or 2; and

R₇ is a carboxyalkyl group containing from about 2 to about 3 carbon atoms;

Examples of suitable alkyl betaines include those compounds of the formula:
B-N⁺R₉R₁₀(CH₂)ₚCO₂⁻
wherein
   B is an alkyl or alkenyl group having from about 8 to about 22, e.g., from about 8 to about 16 carbon atoms;
   R₉ and R₁₀ are each independently an alkyl or hydroxyalkyl group having from about 1 to about 4 carbon atoms; and
   p is 1 or2.

Examples of suitable amidoalkyl betaines include those compounds of the formula:
D-CO—NH(CH₂)_{q}—N⁺R₁₁R₁₂(CH₂)ₘCO₂⁻
wherein
   D is an alkyl or alkenyl group having from about 7 to about 21, e.g. from about 7 to about 15 carbon atoms;
   R₁₁ and R₁₂ are each independently an alkyl or
   Hydroxyalkyl group having from about 1 to about 4 carbon atoms;
   q is an integer from about 2 to about 6; and m is 1 or 2.

One amidoalkyl betaine is cocamidopropyl betaine, available commercially from Goldschmidt Chemical Corporation of Hopewell, Va. under the tradename, “Tegobetaine L7.”

Examples of suitable amidoalkyl sultaines include those compounds of the formula 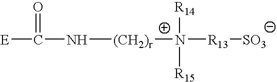
wherein
   E is an alkyl or alkenyl group having from about 7 to about 21, e.g. from about 7 to about 15 carbon atoms;
   R₁₄ and R₁₅ are each independently an alkyl, or hydroxyalkyl group having from about 1 to about 4 carbon atoms;
   r is an integer from about 2 to about 6; and
   R₁₃ is an alkylene or hydroxyalkylene group having from about 2 to about 3 carbon atoms;

Examples of suitable amphophosphate compounds include those of the formula: 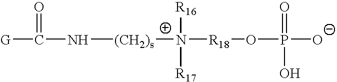
wherein
   G is an alkyl or alkenyl group having about 7 to about 21, e.g. from about 7 to about 15 carbon atoms;
   s is an integer from about 2 to about 6;
   R₁₆ is hydrogen or a carboxyalkyl group containing from about 2 to about 3 carbon atoms;
   R₁₇ is a hydroxyalkyl group containing from about 2 to about 3 carbon atoms or a group of the formula:
      R₁₉—O—(CH₂)ₜ—CO₂⁻
wherein
   R₁₉ is an alkylene or hydroxyalkylene group having from about 2 to about 3 carbon atoms and
   t is 1 or 2; and

R₁₈ is an alkylene or hydroxyalkylene group having from about 2 to about 3 carbon atoms.

Examples of suitable phosphobetaines include those compounds of the formula: 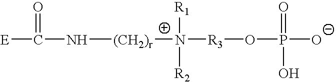
wherein E, r, R₁, R₂ and R₃, are as defined above. In one embodiment, the phosphobetaine compounds are those disclosed in U.S. Pat. Nos. 4,215,064, 4,617,414, and 4,233,192, which are all incorporated herein by reference.

Examples of suitable pyrophosphobetaines include those compounds of the formula:

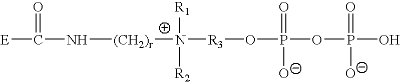

wherein E, r, R₁, R₂ and R₃, are as defined above. In one embodiment, the pyrophosphobetaine compounds are those disclosed in U.S. Pat. Nos. 4,382,036, 4,372,869, and 4,617,414, which are all incorporated herein by reference.

Examples of suitable carboxyalkyl alkylpolyamines include those of the formula: 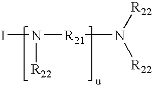
wherein
   I is an alkyl or alkenyl group containing from about 8 to about 22, e.g. from about 8 to about 16 carbon atoms;
   R₂₂ is a carboxyalkyl group having from about 2 to about 3 carbon atoms;
   R₂₁ is an alkylene group having from about 2 to about 3 carbon atoms and
   u is an integer from about 1 to about 4.

The foaming agent may be present in a weight percentage in the composition that is from about 0.01% to about 5%, such as from about 0.5% to about 5%, preferably from about 0.5% to about 4%, more preferably from about 1% to about 4%.

The compositions of the present invention may further comprise any of a variety of additives or other materials used conventionally. For example, the present compositions may also include dyes, fragrances, and other functional ingredients common to skin care compositions, as long as they do not detract from the phase stability, foaming, or deposition of the personal care composition. In general, in order to maintain phase stability, the level of electrolyte (e.g., ionized moieties other than the wetting agent) is maintained or substantially maintained at a relatively low level, such as less than about 2%, such as less than about 0.5% of the total composition.

The present compositions are preferably formulated to be oil-in-water emulsions that are shelf-stable in that the emulsion does not lose phase stability or “break” when kept at standard conditions (22 degrees Celsius, 50% relative humidity) for a week or more after it is made.

The viscosity of the personal care composition is not critical in certain embodiments, although may be a spreadable cream having a viscosity greater than about 5000 centipoise when measured with a Brookfield viscometer with an LVT2 spindle at 12 RPM

Applicants have recognized that the compositions of the present invention may be used advantageously as or in cleansing compositions. The compositions, for example, may be used to remove excess sebum, dirt, oil, makeup, and various foreign matter. The compositions of the present invention deposit various hydrophobic agents, including, for example, emollients and/or various hydrophobic benefit agents as specified above.

In yet another notable embodiment of the invention, the personal care composition may include a sunscreen (e.g. microfine titanium dioxide and Zinc Oxide) and may be applied to the skin to protect the skin from the damaging effects of ultraviolet radiation.

In certain embodiments, the personal care compositions of the present invention are applied to the skin. The skin may, but is not necessarily, pre-moistened with water. The composition may be left on the skin for a period of time such as less than about 30 minutes, preferably less than about 0.5 minutes to less than about 2 minutes, after which it is rinsed such as with water that may be splashed or wiped onto the skin. As a result of the rinsing procedure, portions of the composition that were applied to the skin are removed and other portions remain behind on the skin.

In another aspect, the present invention provides methods of promoting a product by providing a personal care product comprising a composition of the present invention and directing a user to apply the composition to mammalian skin and/or hair to achieve high foaming, high deposition, and/or effective barrier formation.

As used herein, the term “product” refers to a product in finished packaged form. In one embodiment, the package is a container such as a bottle, tube, jar, or other container made from, for example, plastic, metal, glass, combinations thereof, and the like, containing the composition. The product may further contain additional packaging such as a package insert and/or a plastic or cardboard box, or other outer packaging for storing such container.

Any conventional or other means for producing a product comprising a composition of the present invention may be used according to the present invention. In certain preferred embodiments, the product is a personal care product such as, for example, a cleansing product such as a conditioner, soap, facial cleanser, acne treatment, make-up remover, UV protection product, cleansing wipes, creams, gels, lotions, and the like. In light of the teachings herein and knowledge common in the art, those of skill in the art will be readily able to produce a product according to the present invention.

Any suitable means for directing a user to apply the composition of a product of the present invention to the skin, hair, or both, may be used according to the directing step of the present methods. Examples of methods of directing a user include, but are not limited to, written, visual, or verbal statements made on the product, or in stores, magazines, newspaper, radio, television, internet, and the like as advertising and/or marketing for the product. In certain preferred embodiments, the product contains written instructions on the product directing the user to topically apply the composition to the skin and/or hair. Such instructions may be printed on the container, label insert, or on any additional packaging. Preferably, the written, visual, or verbal statements include a description of the foaming and/or high deposition associated with product composition and/or use of the product composition. In certain preferred embodiments, the product contains written description of the high foaming and/or high deposition properties associated with the product composition. Any description suitable to indicate the high foaming and/or high deposition associated with a product comprising a composition of the invention may be used.

### EXAMPLES

### Examples 1-6

The following personal care compositions, Examples 1-6, shown in Tables 1 and 4 and consistent with embodiments of the invention described herein, were prepared. Component amounts in this procedure are given in terms of parts by weight per 100 parts of the final personal care composition. Phase (A): 90.0 parts of water and 0.5 parts paraben were blended at 60° C. until the solution is homogenous. 1.0 parts ammonium acryloyldimethyltaurate/VP Copolymer was slowly added with agitation until the solution is homogenous. Phase (B) In a separate container add 2.0 parts of Mineral Oil was heated to 60° C. Phase B was poured into phase A with rapid agitation and heat was turned off. The emulsion was continually mixed until it was homogenous.

After the emulsion cooled to 40° C., phenoxyethanol was added while continuing to mix. For Examples 2, 3 and 4, disodium laureth sulfosuccinate, cocamidopropyl betaine or fluorobetaine was added (in respective order). For Examples 5 and 6 cocamidopropyl betaine was added. Sufficient water was added to reach a total of 100 parts and the emulsion was mixed for an additional 20 minutes to ensure homogeneity.

The following Deposition Test was performed on various personal care compositions to measure the Deposition Values of the compositions according to the present invention. In addition to compositions consistent with embodiments of the invention described herein, two additional compositions were evaluated: Cetaphil® Gentle Skin Cleanser (Galderma Laboratories, L.P.) and Cetaphil® Gentle Skin Cleanser to which 2% mineral oil was added. The latter was prepared by adding 2 parts mineral oil to 98 parts Cetaphil® Gentle Skin Cleanser.

Five subjects per test sample were used to determine deposition. Deposition was measured by marking a 2 inch square area on both of the subject's forearms. The test site was rinsed in running tap water (85-100 degrees Fahrenheit) for approximately 15 seconds, then gently patted dry with a paper towel. 0.14 ml of the test formula was applied to the test site and massaged in with index finger for 30 seconds staying completely within the 2 inch area. The area was then allowed to stand undisturbed for another 15 seconds. Using a spray bottle, the test area was sprayed 30 times to rinse the formulation, then gently patted dry with a Kim Wipe® towelette, followed by air drying using piped house for one minute. On the other forearm, the same procedure as above was performed except that no test composition was applied to the test area. This served as the baseline. Five measurements per test site were measure to determine the Deposition Valueusing Sebumeter SM 815 manufactured by Courage and Khazaka (Koln, Germany). The Deposition Value data was analyzed for statistical significance utilizing a one-way ANOVA test at a 95% confidence interval and reported in Tables 2 and 5.

The following Foam Test was performed on various personal care compositions to determine the Maximum Foam Volume upon agitation according to the present invention. The procedure was accomplished by adding 0.36 grams calcium chloride and 5.0 grams of the test product to 994.64 grams of deionized water and mixing until homogenous. The mixture was then added to a sample tank of a Sita R-2000 foam tester (commercially available from Future Digital Scientific, Co.; Bethpage, N.Y.). The test parameters were set to repeat three runs (series count=3) of 250 ml sample size (fill volume=250 ml) with nine stir cycles (stir count=9) for a 30 second stir time per cycle (stir time=30 seconds) with the rotor spinning at 1300 RPM (revolution=1300) at a temperature setting of 30° C.±2° C. Foam Volume data was collected at each stir cycle and the average and standard deviation of the three runs was determined. Maximum Foam. Volume was reported for each Example as the value after the ninth stir cycle. The resulting data was analyzed for statistical significance utilizing a one-way ANOVA test at a 95% confidence interval as is reported in Tables 3 and 6.

TABLE 1
Cleansing Compositions

| | Example | Example | Example | Example |
|---|---|---|---|---|
| INCI Name | #1 | #2 | #3 | #4 |
| Ammonium | 1.0 | 1.0 | 1.0 | 1.0 |
| Acryloyldimethyl- | | | | |
| taurate/VP | | | | |
| Copolymer | | | | |
| Glycerin | 5.0 | 5.0 | 5.0 | 5.0 |
| Methyl, Propel Butyl, | 0.5 | 0.5 | 0.5 | 0.5 |
| Ethyl Parables | | | | |
| Mineral Oil | 2.0 | 2.0 | 2.0 | 2.0 |
| Phenoxyethanol | 0.5 | 0.5 | 0.5 | 0.5 |
| Disodium Laureth | | 0.5 | | |
| Sulfosuccinate | | | | |
| Cocamidopropyl | | | 0.5 | |
| Betaine | | | | |
| Fluorobetaine | | | | 0.5 |
| Water | 91.0 | 90.5 | 90.5 | 90.5 |

TABLE 2
Emollient Deposition Results:

| | EMOLLIENT DEPOSITION (AVG ± SD) |
|---|---|
| EXAMPLE | micrograms per square centimeter |
| Comparative Example 1: | 1.25 ± 1.21 |
| Cetaphil ® | |
| Comparative Example 2: | 25.75 ± 13.54* |
| Cetaphil ® + 2% Mineral | |
| Oil | |
| Example #1 | 50.85 ± 16.66** |
| Example #2 | 7.05 ± 6.54 |
| Example #3 | 31.10 ± 18.67* |
| Example #4 | 34.00 ± 14.29* |

*= Statistically significantly greater than Cetaphil ® at 95% CI.
**= Statistically significantly greater than Examples #2, #3, #4, Cetaphil ® and Cetaphil ® + 2% Mineral Oil at 95% CI.

TABLE 3
Maximum Foam Volume Results:

| EXAMPLE | MAXIMUM FOAM VOLUME (MLS) |
|---|---|
| Comparative Example 1: | 409.7 ± 24.13* |
| Cetaphil ® | |
| Comparative Example 2: | 388.3 ± 6.51* |
| Cetaphil ® + 2% Mineral Oil | |
| Example #1 | 0.00 ± 0.00 |
| Example #2 | 770.0 ± 5.57** |
| Example #3 | 694.3 ± 42.77** |
| Example #4 | 731.0 ± 14.38** |

*= Statistically significantly greater than Example #1 at 95% CI.
**= Statistically significantly greater than Example #1, Cetaphil ® and Cetaphil ® + 2% Mineral Oil at 95% CI

TABLE 4
Cleansing Compositions

| | Example | Example |
|---|---|---|
| INCI Name | #5 | #6 |
| Ammonium | 1.0 | 1.0 |
| Acryloyldimethyl- | | |
| taurate/VP | | |
| Copolymer | | |
| Glycerin | 5.0 | 5.0 |
| Methyl, Propel Butyl, | 0.5 | 0.5 |
| Ethyl Parables | | |
| Mineral Oil | 20.0 | 20.0 |
| Phenoxyethanol | 0.5 | 0.5 |
| Cocamidopropyl | 0.5 | 4.0 |
| Betaine | | |
| Water | 72.5 | 69.0 |

TABLE 5
Emollient Deposition Results:

| | EMOLLIENT DEPOSITION (AVG ± SD) |
|---|---|
| EXAMPLE | micrograms per square centimeter |
| Example #5 | 114.90 ± 90.2* |
| Example #6 | 17.96 ± 26.71 |

*= Statistically significantly greater than Example #6 at 95% CI.

TABLE 6
Maximum Foam Volume Results:

| EXAMPLE | MAXIMUM FOAM VOLUME (MLS) |
|---|---|
| Example #5 | 416.3 ± 85.29 |
| Example #6 | 855.3 ± 18.82* |

*= Statistically significantly greater than Example #5 at 95% CI.

As illustrated by the Examples, the compositions of the present invention exhibit significantly higher Deposition Values, Maximum Foam Volumes, and combinations thereof, as compared to conventional cleansers. Certain preferred compositions of the invention exhibit Deposition Values and/or Maximum Foam Volumes that are at least 1.5 to 2 times greater than comparable conventional cleanser compositions. Such surprisingly higher values provide significant advantage to the instant compositions in a variety of uses including as foaming/deposition/cleansing compositions.

What is claimed is:

1. A composition comprising at least one salt-sensitive polymeric emulsifier, at least one hydrophobic agent, and at least one foaming agent, wherein said composition has at least one of the properties selected from the group consisting of a Deposition Value of greater than about 7 micrograms per square centimeter, a Maximum Foam Volume of greater than about 100 mL, and combinations thereof.

2. The composition of claim 1 wherein said composition has a Deposition Value of greater than about 15 micrograms per square centimeter.

3. The composition of claim 1 wherein said composition has a Deposition Value of greater than about 26 micrograms per square centimeter.

4. The composition of claim 1 wherein said composition has a Deposition Value of greater than about 30 micrograms per square centimeter.

5. The composition of claim 1 wherein said composition has a Deposition Value of greater than about 33 micrograms per square centimeter.

6. The composition of claim 1 wherein said composition has a Maximum Foam Volume of greater than about 400 mL.

7. The composition of claim 2 wherein said composition has a Maximum Foam Volume of greater than about 400 mL.

8. The composition of claim 3 wherein said composition has a Maximum Foam Volume of greater than about 400 mL.

9. The composition of claim 4 wherein said composition has a Maximum Foam Volume of greater than about 400 mL.

10. The composition of claim 1 wherein said composition has a Maximum Foam Volume of greater than about 600 mL.

11. The composition of claim 2 wherein said composition has a Maximum Foam Volume of greater than about 600 mL.

12. The composition of claim 3 wherein said composition has a Maximum Foam Volume of greater than about 600 mL.

13. The composition of claim 1 wherein said polymeric emulsifier comprises a compound selected from the group consisting of hydrophobically modified polyacrylic acid, copolymers derived from acrylamidoalkyl sulfonic acid and at least one cyclic N-vinylcarboxamide, copolymers derived from acrylamidoalkyl sulfonic acid and at least one linear N-vinylcarboxamide, copolymers derived from acrylamidoalkyl sulfonic acid and hydrophobically modified methacrylic acid, homopolymers of acrylamidoalkyl sulfonic acid, and mixtures of two or more thereof.

14. The composition of claim 13 wherein said polymeric emulsifier comprises a compound selected from the group consisting of copolymers derived from acrylamidoalkyl sulfonic acid and at least one cyclic N-vinylcarboxamide, copolymers derived from acrylamidoalkyl sulfonic acid and at least one linear N-vinylcarboxamide, and mixtures of two or more thereof.

15. The composition of claim 1 comprising from at least about 0.3 to about 3 weight percent of polymeric emulsifier.

16. The composition of claim 1 wherein said hydrophobic agent comprises a compound selected from the group consisting of mineral oil, petrolatum, silicone oils, and combinations of two or more thereof.

17. The composition of claim 1 wherein said hydrophobic agent comprises a compound selected from the group consisting of anti-acne agents such as salicylic acid and benzoyl peroxide, retinoids such as retinol, skin lifting agents such as alkanolamines including dimethyl amino ethanol (DMAE), skin lightening agents such as soy extracts, anti-inflammatory agents such as tocopherols including vitamin E, extracts of Feverfew, and combinations of two or more thereof.

18. The composition of claim 17 wherein said composition comprises from at least about 0.1 weight percent to about 20 weight percent of hydrophobic agent.

19. The composition of claim 1 wherein said foaming agent comprises a monomeric surfactant compound.

20. The composition of claim 1 wherein said composition comprises from about 0.01 to about 5 weight percent of foaming agent.

21. The composition of claim 1 wherein said composition is a cream cleanser.

22. The composition of claim 1 further comprising a compound selected from the group consisting of titanium dioxide, zinc oxide, and combinations thereof.

23. A method of cleansing the skin comprising applying a composition of claim 1 to the skin.

24. The method of claim 23 further comprising the step of subsequently rinsing the skin with water.

25. A method of promoting a personal care product having high foaming or deposition properties comprising the steps of providing a product comprising a composition of claim 1, and indicating to a user at least one property associated with the product selected from the group consisting of high foaming, high deposition, and combinations thereof.
